(19) United States
(12) Patent Application Publication
(10) Pub. No.: US 2022/0322989 A1
(43) Pub. Date: Oct. 13, 2022

(54) ELECTRODE CONVERSION SYSTEM AND APPARATUS

(71) Applicant: THE TRUSTEES OF COLUMBIA UNIVERSITY IN THE CITY OF NEW YORK, NEW YORK, NY (US)

(72) Inventor: Paul GARCIA, Irvington, NY (US)

(21) Appl. No.: 17/734,579

(22) Filed: May 2, 2022

Related U.S. Application Data
(63) Continuation of application No. PCT/US2020/058550, filed on Nov. 2, 2020.
(60) Provisional application No. 62/928,885, filed on Oct. 31, 2019.

(51) Int. Cl. A61B 5/291; H01B 7/08; A61B 5/262; A61B 5/252; A61B 5/274

(52) U.S. Cl. CPC A61B 5/291; H01B 7/0838; A61B 5/262; A61B 5/252; A61B 5/274; A61B 2562/227

(57) ABSTRACT

An exemplary apparatus for operating with an electroencephalograms apparatus can be provided, which can include, for example a flexible conductor disposed in a shape of a coil, an electrode disposed on a first end of the flexible conductor, and a coupling configuration disposed on a second end of the flexible conductor, where the coupling configuration can be designed to couple the flexible conductor to the electroencephalograms apparatus. The flexible conductor can be stretchable.

### CROSS-REFERENCE TO RELATED APPLICATION(S)

This application relates to and claims priority from U.S. Patent Application No. 62/928,885, filed on Oct. 31, 2019, the entire disclosure of which is incorporated herein by reference.

### FIELD OF THE DISCLOSURE

The present disclosure relates generally to an electrode conversion system and apparatus, and more specifically, to exemplary embodiments of an exemplary electrode conversion system and apparatus for use in, for example, electroencephalograms (“EEG”).

### BACKGROUND INFORMATION

EEG monitoring during anesthesia is currently an optional monitor. There are strong indications that EEG will become a standard monitor for all general anesthesia cases (e.g., predicted in the next 1-3 years). Various medical device companies have “depth of anesthesia” monitors that are FDA approved for titration of drug efficacy intraoperatively (e.g., during anesthesia). Each monitor differs slightly in the information that they use to calculate their “depth of anesthesia” index. It is unlikely that one index will be considered the standard monitor—but more likely that abbreviated frontal EEG, generally speaking 2-6 electrodes, would be suggested, or mandated, as a standard monitor. Each index depends on accurate placement of electrodes. For this reason, each manufacturer sells a standard sensor that is incompatible with other devices for calculation of their proprietary index.

Currently, manufacturers are marketing their devices as superior to the competition based on their index quality (e.g., less susceptible to interference/noise) or accuracy in predicting certain clinical outcomes. The FDA mandates on all of these devices that, if using the index for titration of anesthesia medication, the raw EEG waveform must be available for viewing by the anesthesiologist to evaluate any artifacts. Current trends suggest that the indices are being less emphasized by the anesthesiology community, and that interpretation of the raw EEG waveform is more important for guiding intraoperative decision-making. If some of the electrodes are improperly connected, most devices display an error rather than displaying the EEG traces from properly functioning electrodes. Sometimes the patient's hair, surgery site, or the presence of other forehead devices (i.e., cerebral oximetry, EMG ground) prevent usual placement of electrodes. The sensors currently have limited options in alternative sites for electrode placement, and in some cases where EEG could be very helpful (i.e., neurosurgery), these sensors cannot be used with their current configuration.

The lack of flexibility in electrode configurations limits the use of these devices in many surgeries. Brain surgery and Ear, Nose, and Throat (“ENT”) surgery are typically performed on older individuals at risk for confusion on emergence, and at risk for unnecessarily high administered doses of anesthesia medication. Each presents unique challenges for EEG monitoring with current electrode sensors. Attempts at alternative placement of the sensor are less than ideal because of the stiffness of the sensor and the limitations of the gel electrodes. Almost all Neurosurgeries and ENT cases use computed tomography (“CT”) and magnetic resonance imaging (“MRI”) computer assisted navigation. Sensors can make it difficult to “sync” the patient with their radiologic imaging. Most thyroid/parathyroid and neck surgery place an EMG electrode on the forehead.

FIG. 1 shows images of two different proprietary sensors made by two different manufactures. Such sensors are not reusable, and must be disposed of after each patient use. These sensors can be expensive, and are certainly more expensive than off the shelf standard sensors that are disposable. For example, these single-use proprietary sensors cost approximately $7-$25 per unit. Non-proprietary disposable electrodes are significantly less expensive (e.g., less than $1 for 6 electrodes). However, each proprietary sensor has an authentication mechanism that is required in order to use a sensor with a specific machine provided by a medical device manufacturer. Since standard off the shelf electrodes do not include this authentication mechanism, a standard electrode cannot be readily used with most machines provided by a medical device manufacturer.

Thus, it may be beneficial to provide an exemplary electrode conversion system and apparatus which can overcome at least some of the deficiencies described herein above.

### SUMMARY OF EXEMPLARY EMBODIMENTS

An exemplary apparatus for operating with an electroencephalograms apparatus can be provided, which can include, for example a flexible conductor disposed in a shape of a coil, an electrode disposed on a first end of the flexible conductor, and a coupling configuration disposed on a second end of the flexible conductor, where the coupling configuration can be designed to couple the flexible conductor to the electroencephalograms apparatus. The flexible conductor can be stretchable. The flexible conductor can include a flexible sheath. The electrode can be or can include a needle electrode or a cup electrode.

Additionally, an exemplary apparatus operable with an electroencephalograms (EEG) apparatus can be provided, which can include, for example, a conductor, a first electrode connected to a first end of the conductor, and a coupling configuration connected at a second end of the conductor, where the coupling configuration can be configured to electrically couple the first electrode to a second electrode that can be coupled to the EEG apparatus, and the first end can be opposite to the second end. The coupling configuration can include an adhesive pad configured to adhere to the second electrode. A first snap electrode can be included which can be configured to connect to the second electrode, and the coupling configuration can include a second snap electrode attached to the conductor that can be configured to mechanically and electrically couple to the first snap electrode.

In some exemplary embodiments of the present disclosure, the coupling mechanism can include, for example, a first snap electrode configured to connect to the second electrode. A first cable can include (i) a second snap electrode located on a third end of the first cable and configured to mechanically and electrically couple to the first snap electrode, and (ii) a first connector located at a fourth end of the first cable, where the fourth end is opposite to the third end. A second cable can include (i) a second connector at a fifth end of the second cable and configured to be mechanically and electrically coupled to the first connector, and (ii) a third connector at a sixth end of the second cable, where the sixth end can be opposite to the fifth end, and the third connector can be configured to be coupled to a fourth connector on the conductor.

In certain exemplary embodiments of the present disclosure, the first electrode can be a needle electrode or a cup electrode. The coupling configuration can include a rigid structure, at least one first set of connectors located on a first side of the rigid structure configured to couple to the first electrode, at least one second set of connectors located on a second side of the rigid structure configured to couple to the second electrode, and a plurality of third conductors disposed on the rigid structure, where one of the third conductors can be configured to couple one of the at least one first set of connectors to one of the second set of connectors. The apparatus can include an impedance tuner disposed on the rigid structure configured to tune an impedance of the conductor.

Further, a unitary apparatus can be provided, which can include, for example, a plurality of first connectors configured to interface with a plurality of first electroencephalograms (EEG) electrodes, a plurality of second connectors configured to interface with a plurality of second EEG electrodes, and a plurality of conductors embedded in the unitary apparatus, where each of the conductors can be configured to electrically couple one of the first EEG electrodes to respective one of the second EEG electrodes. The first connectors can be configured to adhesively interface with the plurality of first EEG electrodes. A connection block can be included, which can be configured to have a plurality of conductors from an EEG apparatus crimped thereto, where the plurality of first connectors can be configured to interface with the connection block. A plurality of impedance tuners can be included, where each one of the conductors can include one of the impedance tuners configured to tune a frequency(ies) provided to the EEG apparatus.

In some exemplary embodiments of the present disclosure, the unitary apparatus can include a computer interface(s) configured to receive a modification of at least one of the impedance tuners from a computer(s). The impedance tuners can be configured to be manually modified. The unitary apparatus can have a rigid structure. The unitary apparatus can be or can include a printed circuit board. The plurality of connectors can be embedded on a surface of the unitary apparatus.

Additionally, an apparatus for operating with an electroencephalograms (“EEG:) apparatus can be provided, which can include, for example, a first conductor having a first end, a second conductor having an electrode coupled to a second end thereof, where the second conductor can be different than the first conductor, and a coupling configuration configured to electrically and mechanically couple the first conductor to the second conductor, where the coupling configuration can be configured to facilitate a movement of the second conductor relative to the first conductor such that an electrical distance between the first end and the second end is increased or decreased.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a set of images of prior proprietary EEG sensors;

FIG. 2 is a set of exemplary diagrams illustrating flexible and extendible EEG electrodes according to an exemplary embodiment of the present disclosure as compared to the prior proprietary EEG sensors;

FIG. 3 is an exemplary diagram of an exemplary conversion system which can connect a proprietary sensor to a standard electrode according to an exemplary embodiment of the present disclosure;

FIG. 4 is an exemplary diagram of a further exemplary conversion system from a proprietary sensor to a standard electrode according to an exemplary embodiment of the present disclosure;

FIG. 5 is a further exemplary diagram of the exemplary conversion system shown in FIG. 4, which can convert a proprietary sensor to a standard electrode according to an exemplary embodiment of the present disclosure; and

FIG. 6 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXEMPLARY EMBODIMENTS

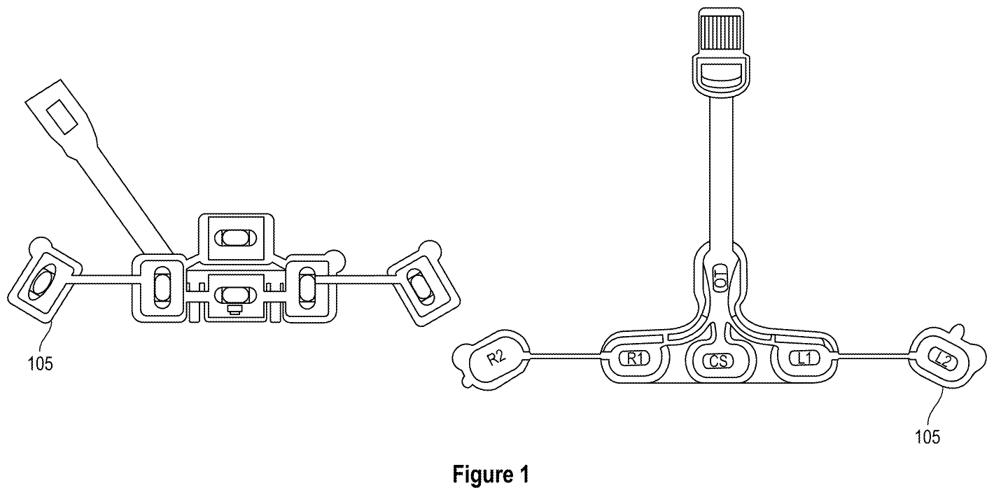

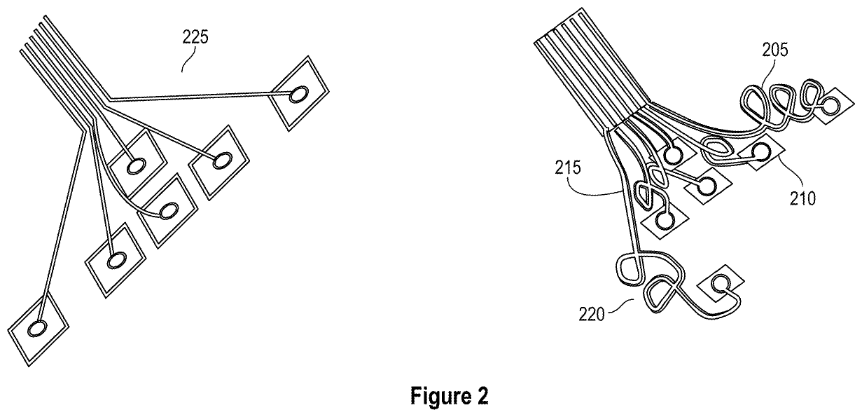

FIG. 2 shows a set of exemplary diagrams of flexible and extendible EEG electrodes 205 according to an exemplary embodiment of the present disclosure as compared to the prior proprietary EEG sensors 225. For example, extendible EEG electrodes 205 can include lower profile sensors 210 with flexible ribbon cables 215 for variable placement of electrodes, which can be used intraoperative EEG monitoring. Prior sensors (e.g., as shown in FIG. 1) generally include flexible plastic ribbon cables with a proprietary connector to their device. These sensors are typically placed across the forehead over locations F1/2, Fz, Fpz/Cz, F7/F8. However, the relative location of each electrode in the proprietary sensor (e.g., as shown in FIG. 1) cannot be easily moved. For example, as shown in FIG. 1, there may be some variability in the placement of the sensors 105. However, such variability is very limited. In order to address at least such limitation, as shown in FIG. 2, the exemplary apparatus can include flexible cables 215 that can facilitate various placements of each of the electrodes. Thus, the medical professional can modify the location of each sensor 210, which can be based on each patient and/or on the procedure being performed. Flexible cables 215 can be stretchable. For example, each flexible cable 215 can be composed of or include a stretchable metal conductor surrounded by a stretchable sheath. Alternatively or in addition, flexible cable 215 can be formed into a coil structure 220, which can facilitate the electrode to be placed at a certain distance when the cable is at rest, and in various other positions when the cable is stretched (e.g., the coil structure is stretched). By facilitating the medical professional to determine the placement of the electrode, various improvements in prior systems can be achieved.

Additionally, the exemplary EEG electrodes can be included on one or more conductors that are configured to change the length of the electrical connection (e.g., an electrical distance) between the conductor and the EEG apparatus. The length of the electrical connection of the electrical distance is a distance that an electron travels from one point to another. For example, two separate wires can be coupled together with a mechanism which allows one wire to move relative to the other wire in order to extend the length of the two wires combined together, and thus increase or decrease the distance an electron would travel from one end to the other end. Alternatively, a stretchable conductor can be used that can facilitate the lengthening of a single conductor. Additionally, the conductor can be wound in a wheel mechanism which can facilitate the conductor to be wound around. The conductor can then be extended or retracted as need depending on the desired position of the electrode.

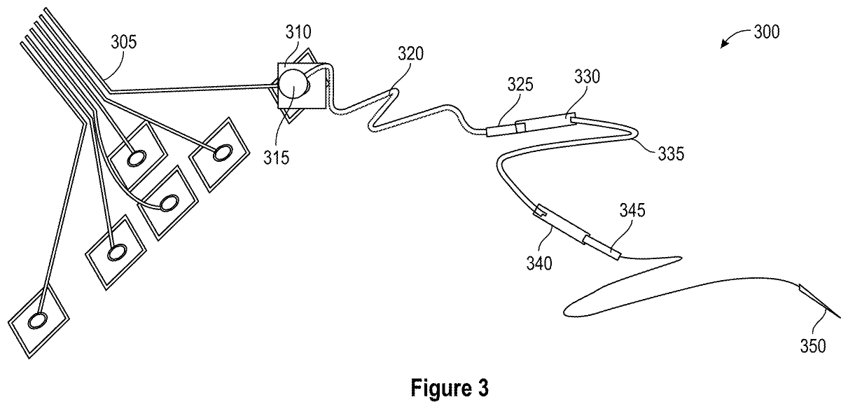

FIG. 3 shows an exemplary diagram of an exemplary system 300, which can connect a proprietary sensor 305 to a standard electrode 350 according to an exemplary embodiment of the present disclosure. Without using the exemplary system 300, according to the conventional operation, the proprietary sensor 305 is placed directly on the patient's forehead, and the sensor 305 is then disposed of after each use. However, the sensor 305 suffers from the same issues described above regarding the limited placement of each electrode. As shown in FIG. 3, instead, a snap electrode 310, which can be disposable, can be placed on one of the electrodes on sensor 305. Snap electrode 310 can facilitate coupling to the standard electrode 350.

For example, a female snap electrode cable 320 can be coupled to snap electrode 310. Female snap electrode cable 320 can include a snap electrode 315 on one end, which can be coupled to snap electrode 310, and a connector 325 on the other end (e.g., an RS 232 connector). A male to male cable 335 can be provided, having one end that can include a connector 330, which can be coupled to connector 325 of cable 320. The other end of cable 335 can include a connector 340, which can be coupled to a standard electrode 350 (e.g., a cup electrode or a needle electrode), using connector 345. Alternatively, a single electrode cable can be provided, which can have a snap electrode on one end, and a cup electrode or a needle electrode on the other end. Further, instead of including a single cable with a snap electrode at one end, the single cable can have an adhesive pad at one end configured to interface with the electrode attached to sensor 305.

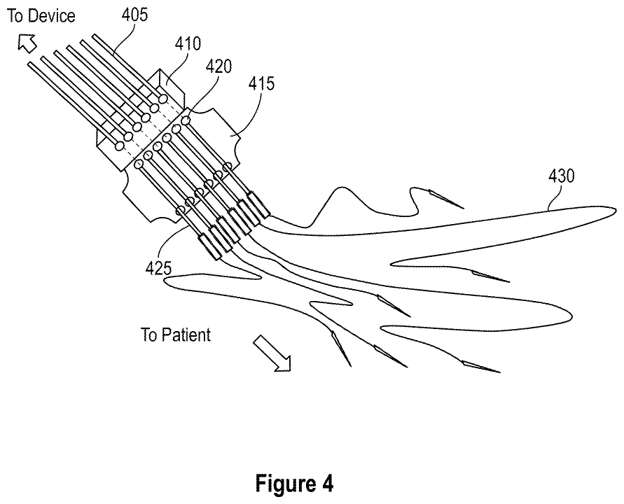

FIG. 4 shows an exemplary diagram of a further exemplary conversion system from a proprietary sensor to a standard electrode according to an exemplary embodiment of the present disclosure. For example, as shown in FIG. 4, the wiring 405 attached to each electrode on a proprietary sensor can be spliced, and then connected to a connection block 410. A conversion device 415 can be included, which can be or can include a printed circuit board. Conversion device 415 can include a set of connectors 420, which can be configured to couple directly with connection block 410. Conversion device 415 can also include a set of standard electrode connectors 425, which can be configured to couple to a standard electrode 430, which can be a cup electrode or a needle electrode.

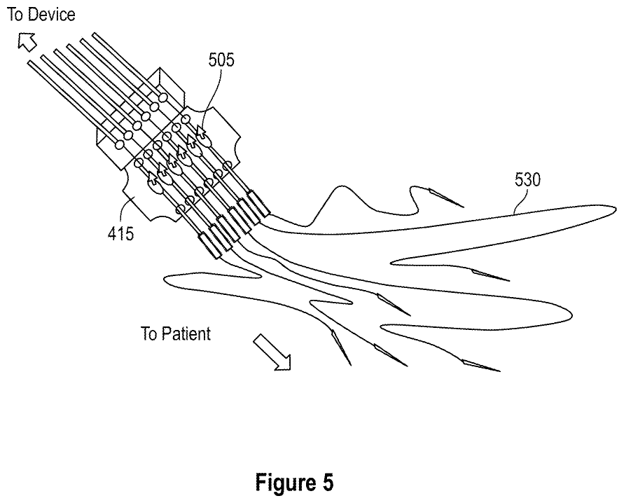

FIG. 5 shows a further exemplary diagram of the exemplary conversion system shown in FIG. 4, which can convert a proprietary sensor to a standard electrode according to an exemplary embodiment of the present disclosure. For example, each proprietary sensor system can operate under different impedance conditions. Exemplary conversion device 415 can include an impedance tuner 505, which can facilitate tuning of the impedance to match the specific proprietary sensor being connected to. For example, various exemplary transformers, resistive networks, and filters, either alone or in combination with one another, can be used to tune the impedance. Each impedance device can be externally accessible (e.g., by hand or by computer interface) to tune the impedance.

In one exemplary operating example, the medical professional can connect the conversion device 415 to the proprietary sensor, and then place the standard electrodes 530 on the patient. The medical professional can then activate the EEG apparatus, and view the output on a display. The display can provide the status of each signal from each electrode. The signal may be impaired depending on the tuning of the impedance level. The medical professional can then modify the impedance level for each electrode until the correct impedance level is reached. This can be based on feedback provided on the display, which can be provided in the form of various colors (e.g., red for no match, orange for close match, and green for match, although other color schemes can be used). Alternatively, a number system can be provided, which can facilitate feedback as to how to tune the impedance to match the correct impedance (e.g., 100 being the correct impedance level, and anything above or below 100 would tune the impedance in a particular direction, although different numbering schemes can be used).

As discussed herein, the impedance can be tuned using a computer interface. For example, the impedance tuner can be a field-programmable gate array (“FPGA”), which can be connected to a computer for tuning. The medical professional can connect a computer to conversion device 415 and set an impedance level. This can be based on the requirements of the proprietary sensor. For example, the medical professional can select the manufacturer of the proprietary sensor. The computer can then automatically program the FPGA based on the target impedance level for the specific proprietary sensor. The computer can also monitor the impedance level once all of the electrodes have been placed on the patient, and can automatically adjust the impedance level based on various patient parameters.

As discussed herein, exemplary conversion device 415 can be configured to be connected to connection block 410, which can have cables 405 spliced and connected thereto. However, conversion device 415 can alternatively include a plurality of connectors, which can be configured to connect directly to each electrode of a proprietary sensor without splicing the proprietary sensor. Thus, the electrodes of the proprietary sensor would not need to be cut. They can simply be placed directly on one end of conversion device 415, with the other end of conversion device 415 have a plurality of standard electrode connectors. This exemplary conversion system can also include impedance tuning as discussed above.

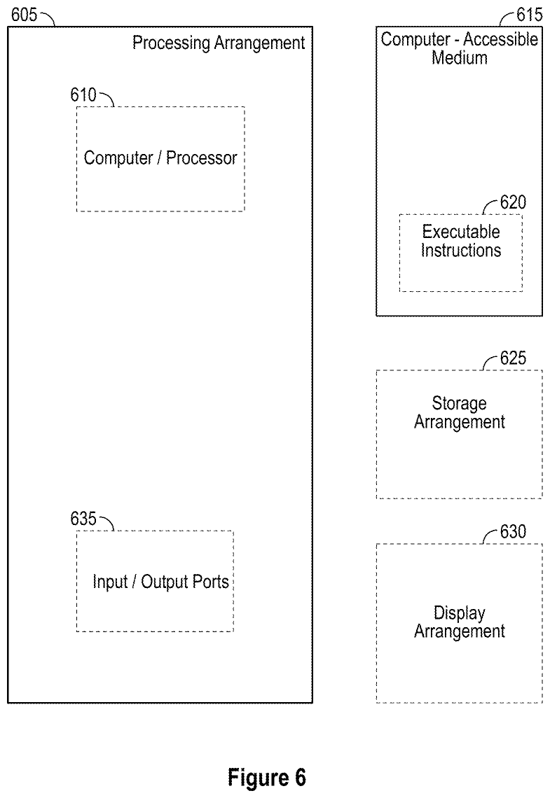

FIG. 6 shows a block diagram of an exemplary embodiment of a system according to the present disclosure. For example, exemplary procedures in accordance with the present disclosure described herein can be performed by a processing arrangement and/or a computing arrangement 602. Such processing/computing arrangement 602 can be, for example entirely or a part of, or include, but not limited to, a computer/processor 604 that can include, for example one or more microprocessors, and use instructions stored on a computer-accessible medium (e.g., RAM, ROM, hard drive, or other storage device).

As shown in FIG. 6, for example a computer-accessible medium 606 (e.g., as described herein above, a storage device such as a hard disk, floppy disk, memory stick, CD-ROM, RAM, ROM, etc., or a collection thereof) can be provided (e.g., in communication with the processing arrangement 602). The computer-accessible medium 606 can contain executable instructions 608 thereon. In addition or alternatively, a storage arrangement 610 can be provided separately from the computer-accessible medium 606, which can provide the instructions to the processing arrangement 602 so as to configure the processing arrangement to execute certain exemplary procedures, processes and methods, as described herein above, for example.

Further, the exemplary processing arrangement 602 can be provided with or include an input/output arrangement 614, which can include, for example a wired network, a wireless network, the internet, an intranet, a data collection probe, a sensor, etc. As shown in FIG. 6, the exemplary processing arrangement 602 can be in communication with an exemplary display arrangement 612, which, according to certain exemplary embodiments of the present disclosure, can be a touch-screen configured for inputting information to the processing arrangement in addition to outputting information from the processing arrangement, for example. Further, the exemplary display 612 and/or a storage arrangement 610 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. An apparatus for operating with an electroencephalograms apparatus, comprising:
at least one flexible conductor disposed in a shape of a coil;
an electrode disposed on a first end of the at least one flexible conductor; and
a coupling configuration disposed on a second end of the at least one flexible conductor, wherein the coupling configuration is designed to couple the at least one flexible conductor to the electroencephalograms apparatus.

2. The apparatus of claim 1, wherein the at least one flexible conductor is stretchable.

3. The apparatus of claim 2, wherein the at least one flexible conductor includes a flexible sheath.

4. The apparatus of claim 1, wherein the electrode includes at least one of a needle electrode or a cup electrode.

5. An apparatus operable with an electroencephalograms (EEG) apparatus, comprising:
a conductor;
a first electrode connected to a first end of the conductor; and
a coupling configuration connected at a second end of the conductor, wherein the coupling configuration is configured to electrically couple the first electrode to a second electrode that is coupled to the EEG apparatus, and wherein the first end is opposite to the second end.

6. The apparatus of claim 5, wherein the coupling configuration includes an adhesive pad configured to adhere to the second electrode.

7. The apparatus of claim 5, further comprising a first snap electrode configured to connect to the second electrode, wherein the coupling configuration includes a second snap electrode attached to the conductor that is configured to mechanically and electrically couple to the first snap electrode.

8. The apparatus of claim 5, wherein the coupling mechanism includes:
a first snap electrode configured to connect to the second electrode;
a first cable which includes (i) a second snap electrode located on a third end of the first cable and configured to mechanically and electrically couple to the first snap electrode, and (ii) a first connector located at a fourth end of the first cable, wherein the fourth end is opposite to the third end;
a second cable includes (i) a second connector at a fifth end of the second cable and configured to be mechanically and electrically coupled to the first connector, and (ii) a third connector at a sixth end of the second cable, wherein the sixth end is opposite to the fifth end, and wherein the third connector is configured to be coupled to a fourth connector on the conductor.

9. The apparatus of claim 5, wherein the first electrode includes at least one of a needle electrode or a cup electrode.

10. The apparatus of claim 5, wherein the coupling configuration includes:
a rigid structure;
at least one first set of connectors located on a first side of the rigid structure and configured to couple to the first electrode;
at least one second set of connectors located on a second side of the rigid structure and configured to couple to the second electrode; and
a plurality of third conductors disposed on the rigid structure, wherein one of the third conductors is configured to couple one of the at least one first set of connectors to one of the second set of connectors.

11. The apparatus of claim 10, further comprising at least one impedance tuner disposed on the rigid structure and configured to tune an impedance of the conductor.

12. A unitary apparatus, comprising:
a plurality of first connectors configured to interface with a plurality of first electroencephalograms (EEG) electrodes;
a plurality of second connectors configured to interface with a plurality of second EEG electrodes; and
a plurality of conductors embedded in the unitary apparatus, wherein each of the conductors is configured to electrically couple one of the first EEG electrodes to respective one of the second EEG electrodes.

13. The apparatus of claim 12, wherein the first connectors are configured to adhesively interface with the plurality of first EEG electrodes.

14. The apparatus of claim 12, further comprising a connection block configured to have a plurality of conductors from an EEG apparatus crimped thereto, wherein the plurality of first connectors are configured to interface with the connection block.

15. The apparatus of claim 12, further comprising a plurality of impedance tuners, wherein each one of the conductors includes one of the impedance tuners configured to tune at least one frequency provided to the EEG apparatus.

16. The apparatus of claim 15, wherein the unitary apparatus includes at least one computer interface configured to receive a modification of at least one of the impedance tuners from at least one computer.

17. The apparatus of claim 15, wherein the impedance tuners are configured to be manually modified.

18. The apparatus of claim 12, wherein the unitary apparatus has a rigid structure.

19. The apparatus of claim 16, wherein the unitary apparatus is a printed circuit board.

20. The apparatus of claim 12, wherein the plurality of connectors are embedded on a surface of the unitary apparatus.

21. An apparatus for operating with an electroencephalograms (EEG) apparatus, comprising:
a first conductor having a first end;
a second conductor having an electrode coupled to a second end thereof, wherein the second conductor is different than the first conductor; and
a coupling configuration configured to electrically and mechanically couple the first conductor to the second conductor, wherein the coupling configuration is configured to facilitate a movement of the second conductor relative to the first conductor such that an electrical distance between the first end and the second end is increased or decreased.

22. The apparatus of claim 21, wherein the coupling configuration is configured to facilitate a movement of the second conductor relative to the first conductor of each of the first and second conductors such that an electrical distance between the first end and the second end of each of the conductors is increased or decreased in different directions from one another.

23. The apparatus of claim 1, wherein the at least one flexible conductor includes at least two flexible conductors, and wherein each of the at least two flexible conductors:
is disposed in a shape of a coil,
has the electrode provided away from the EEG apparatus that is unconnected to the electrode of another one of the at least two flexible conductors, and
is separated from another one of the at least two flexible conductors at a distance therefrom.
